(19) United States
(12) Patent Application Publication
(10) Pub. No.: US 2006/0216300 A1
(43) Pub. Date: Sep. 28, 2006

(54) Recombinant antibodies and compositions and methods for making and using the same

(75) Inventors: Douglas Craig Hooper, Medford, NJ (US); Bernhard Dietzschold, Newtown Square, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Thomas Jefferson University, Philadelphia, PA (US)

(21) Appl. No.: 10/560,299

(22) PCT Filed: Jun. 10, 2004
(86) PCT No.: PCT/US04/18613

§ 371 Date: Feb. 13, 2006

Related U.S. Application Data
(63) Continuation-in-part of application No. 10/461,148, filed on Jun. 13, 2003.

(51) Int. Cl. A61K 39/42; G01N 33/53

(52) U.S. Cl. 424/159.1; 435/7.1

(57) ABSTRACT

Methods and compositions are provided for the use of rabies virusneutralizing antibodies for postexposure prophylactic treatment of subjects exposed to rabies virus. Compositions comprising mixtures of rabies virusneutralizing antibodies, as well as nucleotide and amino acid sequences encoding these antibodies, can be used in the treatment of subjects exposed to rabies virus. The invention also provides methods of producing recombinant rabies virus-neutralizing human antibodies in mammalian cells using recombinant expression vectors.

### REFERENCE TO GOVERNMENT GRANT

The invention described herein was supported in part using funds obtained from the U.S. Government (National Institutes of Health Grant AI450079). The U.S. Government has certain rights in this invention.

### FIELD OF THE INVENTION

The present invention relates to antibodies, including the nucleic acid and amino acid sequences of human rabies virus-neutralizing antibodies, and their production using recombinant expression vectors. More specifically, the invention relates to the use of a mixture of antibodies directed against rabies virus which can be used for prophylactic treatment following exposure of a subject to a rabies virus.

### BACKGROUND OF THE INVENTION

Rabies is an acute, neurological disease caused by infection of the central nervous system with rabies virus, a member of the Lyssavirus genus of the family Rhabdoviridae. Of great historical significance due to its antiquity and the horrific nature of the disease, rabies virus continues to be an important threat of human and veterinary infection because of extensive reservoirs in diverse species of wildlife. Throughout much of the world, distinct variants of rabies virus are endemic in particular terrestrial animal species, with relatively little in common between them. While several islands, including the United Kingdom, Australia, Japan, and numerous islands are free of terrestrial rabies, rabies and rabies-related viruses associated with bats have recently been identified in the UK and Australia.

Whereas antibodies can provide immediate immunity, passive immunization has been widely used for the therapy of infectious diseases, including rabies (Casadeval, A., Emerg. Infect. Dis. 2002, 8:833-41). According to World Health Organization (WHO) guidelines, category 3 exposures to rabies, which are defined as either single or multiple transdermal bites or contamination of mucous membranes with saliva of a rabid animal, require rabies postexposure prophylaxis (PEP) (Human rabies prevention-United States, 2000: Recommendations of the Advisory Committee on Immunization Practices. MMWR 2000). Rabies postexposure prophylaxis includes administration of both vaccine and anti-rabies immunoglobulin (RIG). At present, RIG for postexposure prophylaxis is prepared from the serum samples of either rabies virus-immune humans (human RIG [HRIG]) or rabies virus-immune horses (equine RIG [ERIG]). Because of the potential adverse effects (e.g., anaphylactic shock) associated with the use of ERIG, only HRIG is used in the United States, and the people who have been exposed to rabid animals (˜39,000 persons annually) receive HRIG in addition to rabies virus (RV) vaccine (Human rabies prevention-United States, 2000: Recommendations of the Advisory Committee on Immunization Practices. MMWR 2000). In developing countries, however, <1% of all PEP includes administration of RIG, because HRIG and ERIG either are not available in sufficient quantities or, as is especially the case for HRIG, are too expensive (Human rabies prevention-United States, 1999: Recommendations of the Advisory Committee on Immunization Practices (ACIP). MMWR Recomm. Rep. 1999, 48:1-21). Furthermore, some animal-protection groups condemn the rearing of animals for serum production (Human rabies prevention-United States, 1999: Recommendations of the Advisory Committee on Immunization Practices (ACIP). MMWR Recomm. Rep. 1999 48:1-21). In addition, the possibility of contamination of HRIG by known or unknown pathogens is a concern of the regulatory authorities. The limited worldwide supply of cost-effective and safe RIG could be overcome by the production of human RV-neutralizing monoclonal antibodies (MAbs) that are used in high-yield expression systems (e.g., those employing transfectomas) in conjunction with bioreactor technology.

Once an individual has been exposed to rabies virus, the disease can readily be prevented by appropriate treatment, which includes both passive and active immunization (Human rabies prevention—United States, 2000: Recommendations of the Advisory Committee on Immunization Practices. MMWR 2000). The passive administration of antibody is believed to be important in neutralizing the virus at the site of entry, as well as in interfering with spread of the virus to the central nervous system (CNS) (Dietzschold et al., Proc. Natl. Acad. Sci. USA 1992, 89:7252-6). These two actions likely allow time for the development of active immunity to the virus, immunity that ultimately results in the clearance of the virus.

Administration of RV-neutralizing mouse or human monoclonal antibodies (MAbs) has been shown to be efficient in postexposure prophylaxis of rodents (WHO consultation on a monoclonal antibody cocktail for rabies post exposure treatment, WHO, 23-24 May 2002; Schumacher et al., J. Clin. Invest. 1989 84:971-5; Dietzschold et al., J. Virol. 1990 64:3087-90). With advances in the technology for production of human monoclonal antibodies, it may be unnecessary to consider the use of mouse monoclonal antibodies in humans. Using mouse monoclonal antibodies in humans may be problematic, because they are immunogenic in humans and their half-life could be limited, possibly leading to either serum sickness or anaphylactic shock. Moreover, the interactions between human monoclonal antibody and Fc receptors are likely to be more appropriate than would be those between comparable mouse reagents.

Currently, antibody prepared from pooled human serum or from immunized horses is utilized to treat subjects. However, neither of these reagents is readily available, entirely safe, or consistent in their biological activity.

There is a need for new methods of postexposure prophylactic treatment of individuals exposed to rabies virus. There is also a need for new methods of treating subjects who are at risk of being exposed to rabies virus. The present invention satisfies these needs.

### SUMMARY OF THE INVENTION

The invention provides for the treatment and prevention of rabies with a mixture of rabies virus-neutralizing antibodies. The invention also provides recombinant rhabdovirus expression systems comprising nucleic acids encoding anti-rabies antibodies, and compositions for and methods of producing such antibodies in mammalian cells.

Recombinant rabies virus-neutralizing human antibodies provided herein include the human antibodies SOJA, SOJB, and SO57. These three antibodies are also referred to as MAb JA, MAb JB.1 and MAb 57, respectively.

The invention thus provides a pharmaceutical composition comprising a pharmaceutically acceptable carrier and at least two rabies virus-neutralizing human antibodies, wherein at least one of the at least two antibodies is selected from the group consisting of:

a) an antibody comprising an antibody light chain having the amino acid sequence SEQ ID NO:2 or a sequence that is substantially homologous to SEQ ID NO:2, and an antibody heavy chain having the amino acid sequence SEQ ID NO:1 or a sequence that is substantially homologous to SEQ ID NO:1;

b) an antibody comprising an antibody light chain having the amino acid sequence SEQ ID NO:6 or a sequence that is substantially homologous to SEQ ID NO:6, and an antibody heavy chain having the amino acid sequence SEQ ID NO:4 or a sequence that is substantially homologous to SEQ ID NO:4; and

c) an antibody comprising an antibody light chain having the amino acid sequence SEQ ID NO:7 or a sequence that is substantially homologous to SEQ ID NO:7, and an antibody heavy chain having the amino acid sequence SEQ ID NO:9 or a sequence that is substantially homologous to SEQ ID NO:9.

The invention provides a method of treating or preventing a rabies virus infection in a subject in need of such treatment. The method comprises administering to the subject an effective amount of at least two recombinant rabies virus-neutralizing human antibodies, wherein the antibodies are selected from the group consisting of:

a) an antibody comprising an antibody light chain having the amino acid sequence SEQ ID NO:2 or a sequence that is substantially homologous to SEQ ID NO:2, and an antibody heavy chain having the amino acid sequence SEQ ID NO:1 or a sequence that is substantially homologous to SEQ ID NO:1;

b) an antibody comprising an antibody light chain having the amino acid sequence SEQ ID NO:6 or a sequence that is substantially homologous to SEQ ID NO:6, and an antibody heavy chain having the amino acid sequence SEQ ID NO:4 or a sequence that is substantially homologous to SEQ ID NO:4; and

c) an antibody comprising an antibody light chain having the amino acid sequence SEQ ID NO:7 or a sequence that is substantially homologous to SEQ ID NO:7, and an antibody heavy chain having the amino acid sequence SEQ ID NO:9 or a sequence that is substantially homologous to SEQ ID NO:9.

In one embodiment, at least two, and more preferably three, of the antibodies are selected from the aforementioned group.

In one embodiment, at least three different recombinant rabies virus-neutralizing human antibodies are administered.

In one embodiment, antibodies are administered separately as at least two different compositions.

In one embodiment, the recombinant rabies virus-neutralizing human antibodies exhibit neutralizing activity against at least two or more rabies viruses.

In one aspect of the invention, the different recombinant rabies virus-neutralizing human antibodies are administered in approximately equimolar concentrations.

In one embodiment, a mixture of recombinant rabies virus-neutralizing human antibodies is administered, wherein each recombinant rabies virus-neutralizing human antibody is administered in approximately equal amounts of protein with other recombinant rabies virus-neutralizing human antibodies being administered. In one aspect, the amount of recombinant rabies virus-neutralizing human antibodies administered is between about 0.001 mg/kg body weight and about 100 mg/kg body weight. In another aspect of the invention, the amount of recombinant rabies virus-neutralizing human antibodies administered is between about 0.01 mg/kg body weight and about 60 mg/kg body weight.

In another embodiment, the amount of recombinant rabies virus-neutralizing human antibodies administered is based on the rabies virus-neutralizing activity of the mixture of recombinant rabies virus-neutralizing human antibodies. In one aspect, the mixture has between about 1 IU/kg body weight and about 50 IU/kg body weight.

In one embodiment, the invention provides a method for treatment of a subject by administering a mixture of recombinant rabies virus-neutralizing antibodies wherein the virus treated is a street rabies virus. In some embodiments, the virus is selected from the group consisting of silver-haired bat rabies virus, coyote street rabies virus/Mexican dog rabies virus, and dog rabies virus.

In another embodiment, the invention provides a method for treatment of a subject by administering a mixture of recombinant rabies virus-neutralizing antibodies wherein the virus treated is a fixed rabies virus.

The treated subject is preferably a human being.

In one embodiment, the invention provides a recombinant rhabdovirus expression vector, comprising a nucleic acid sequence encoding a vesicularstomatitisvirus glycoprotein sequence, and further comprising a nucleic acid comprising a nucleic acid sequence encoding an antibody light chain and an antibody heavy chain of a recombinant rabies virus-neutralizing human antibody. In another embodiment, the nucleic acid sequence encodes the light chain of a rabies virus-neutralizing human antibody, but not the heavy chain. In yet another embodiment, the nucleic acid sequence encodes the heavy chain of a rabies virus-neutralizing human antibody, but not the light chain. In one aspect, the antibody is the SOJA monoclonal antibody. In another aspect, the antibody is the SOJB monoclonal antibody. In yet another aspect, the antibody is the SO57 monoclonal antibody. In one aspect, the nucleic acid encodes only an antibody light chain. In another aspect, the nucleic acid encodes only a heavy chain.

In the practice of the invention, a host mammalian cell is provided which comprises a recombinant rhabdovirus expression vector of the invention. In one embodiment of the invention, the host mammalian cell is a BSR cell, a baby hamster kidney cell, a VERO cell, or a chinese hamster ovary cell.

The invention also provides a method of producing a recombinant rabies virus-neutralizing human antibody in a host mammalian cell using a recombinant rhabdovirus expression vector of the invention. The method comprises infecting a mammalian cell with a recombinant rhabdovirus expression vector of the invention, comprising a nucleic acid comprising a nucleic acid sequence encoding an antibody light chain and an antibody heavy chain of a recombinant rabies virus-neutralizing human antibody, and culturing the mammalian cell under conditions which allow expression of the antibody. In one aspect, the antibody is the SOJA monoclonal antibody. In another aspect, the antibody is the SOJB monoclonal antibody. In yet another aspect, the antibody is the SO57 monoclonal antibody. In one aspect, the nucleic acid encodes only an antibody light chain. In another aspect, the nucleic acid encodes only a heavy chain.

In one embodiment of the invention, the mammalian cell producing a recombinant rabies virus-neutralizing human antibody is a BSR cell, a baby hamster kidney cell, a VERO cell, or a chinese hamster ovary cell.

### Abbreviations and Short Forms

The following abbreviations and short forms are used in this specification.

“BHK” means baby hamster kidney.

“BSR” means a subclone of BHK cells.

“CHO” means chinese hamster ovary.

“CNS” means central nervous system.

“COSRV” means canine rabies virus variant.

“CVS” means challenge-virus standard.

“DRV-4” means dog rabies virus 4.

“EBV-2” means European bat virus 2.

“ED₅₀” means effective dose at which 50% of treated animals are protected.

“ERIG” means equine rabies immunoglobulin.

“G” means glycoprotein.

“GSP” means gene-specific promoter.

“HRIG” means human rabies immunoglobulin.

“huMAb” means human monoclonal antibody.

“Ig H” means immunoglobulin heavy chain.

“Ig L” means immunoglobulin light chain.

“IU” means international unit.

“MAb” means monoclonal antibody.

“MIC LD₅₀” means the dose of virus that kills 50 percent of mice infected intra-cranially, i.e., mouse intra-cranial LD₅₀.

“MOI” means multiplicity of infection.

“PCR” means polymerase chain reaction.

“PEP” means post-exposure prophylaxis.

“RIG” means rabies immunoglobulin.

“rhuMAb” means recombinant human monoclonal antibody.

“RhV” means rhabdovirus vector.

“RV” means rabies virus.

“SHBRV” means silver-haired-bat rabies virus.

“VNA” means virus-neutralization antibody.

“VSV” means vesicularstomatitisvirus.

“WHO” means World Health Organization.

### Definitions

The definitions used in this application are for illustrative purposes and do not limit the scope of the invention.

The articles “a” and “an” are used herein to refer to one or to more than one (i.e., to at least one) of the grammatical object of the article. By way of example, “an element” means one element or more than one element.

As used herein, each “amino acid” is represented by the full name thereof, by the three letter code corresponding thereto, or by the one-letter code corresponding thereto, as indicated in the following table:

| | Three-Letter | One-Letter |
|---|---|---|
| Full Name | Code | Code |
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty L-amino acids commonly found in naturally occurring peptides. “Nonstandard amino acid residues” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change a peptide's circulating half life without adversely affecting activity of the peptide. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

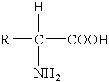

Amino acids are classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino-and carboxy-terminal groups, although not specifically shown, will be understood to be in the form they would assume at physiologic pH values, unless otherwise specified.

“Antibody,” as used herein, includes polyclonal and monoclonal antibodies; recombinant antibodies; and chimeric, single chain, humanized antibodies, and human antibodies. “Antibody” includes not only intact antigen binding immunoglobulin molecules, but also fragments thereof which bind antigen, such as Fv, Fab, Fab′, and F(ab′)₂ fragments, single chain Fv, or the product of an immunoglobulin expression library.

An “antibody heavy chain,” as used herein, refers to the larger of the two types of polypeptide chains present in all antibody molecules.

An “antibody light chain,” as used herein, refers to the smaller of the two types of polypeptide chains present in all antibody molecules.

“Biologically active,” as used herein with respect to rabies virus-neutralizing recombinant antibodies, refers to the ability to function as neutralizers of rabies virus activity.

As used herein, an “effective amount” or “therapeutically effective amount” of rabies virus-neutralizing antibodies, is an amount sufficient to inhibit progression of a rabies virus infection in a subject exposed to rabies or to prevent progression in a subject at risk of exposure to a rabies virus.

The term “expression,” as used with respect to a rabies virus-neutralizing antibody mRNA, refers to transcription of a rabies virus-neutralizing heavy or light chain nucleic acid sequence, resulting in synthesis of rabies virus-neutralizing antibody mRNA. “Expression,” as used with respect to a rabies virus-neutralizing antibody, refers to translation of a rabies virus-neutralizing antibody mRNA, resulting in synthesis of a rabies virus-neutralizing antibody.

“Homologous” as used herein, refers to the subunit sequence similarity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. By way of example, the DNA sequences 3′ATTGCC5′ and 3′TATGGC are 50% homologous.

As used herein, a “subunit” of a nucleic acid molecule is a nucleotide, and a “subunit” of a polypeptide is an amino acid.

As used herein, “homology” is used synonymously with “identity.”

The term “inhibit,” as used herein, means to suppress or block an activity or function by at least ten percent relative to a control value. Preferably, the activity is suppressed or blocked by 50% compared to a control value, more preferably by 75%, and even more preferably by 95%.

“Isolated” means altered or removed from the natural state through the actions of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

“Neutralize,” as used herein with respect to a rabies virus, means to reduce or inhibit progression of a rabies virus infection in a subject exposed to rabies or to reduce or prevent progression in a subject at risk of exposure to a rabies virus.

A “nucleic acid” refers to a polynucleotide and includes poly-ribonucleotides and poly-deoxyribonucleotides.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids which can comprise a protein's or peptide's sequence.

“Pharmaceutically acceptable” means physiologically tolerable, for either human or veterinary applications.

As used herein, “pharmaceutical compositions” include formulations for human and veterinary use.

A “preventive” or “prophylactic” treatment is a treatment administered to a subject who does not exhibit signs, or exhibits only early signs, of rabies virus exposure or infection. A prophylactic or preventative treatment is administered for the purpose of decreasing the risk of developing pathology associated with rabies virus infection.

“Rabies virus-associated disorder,” as used herein, refers to a disorder in which there is an association between the presence of a rabies virus and clinical signs of rabies.

“Rabies virus-neutralizing,” as used herein with respect to recombinant human antibodies, refers to an antibody or mixture of antibodies which exhibits the ability to reduce the extent to which a rabies virus infects a cell or causes rabies. “Rabies virus-neutralizing” is used interchangeably with “Rabies virus-neutralizing activity.”

A “sample,” as used herein, refers to a biological sample from a subject, including normal tissue samples, blood, saliva, feces, or urine. A sample can also be any other source of material obtained from a subject which contains a compound or cells of interest.

A “subject,” as used herein, can be a human or non-human animal. Non-human animals include, for example, livestock and pets, such as ovine, bovine, porcine, canine, feline and murine mammals, as well as reptiles, birds and fish. Preferably, the subject is a human.

“Substantially purified” refers to a peptide or nucleic acid sequence which is substantially homogenous in character due to the removal of other compounds (e.g., other peptides, nucleic acids, carbohydrates, lipids) or other cells originally present. “Substantially purified” is not meant to exclude artificial or synthetic mixtures with other compounds, or the presence of impurities which do not interfere with biological activity, and which may be present, for example, due to incomplete purification, addition of stabilizers, or formulation into a pharmaceutically acceptable preparation.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially similar nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial similarity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C. with washing in 1× SSC, 0.1% SDS at 50° C.; preferably 7% SDS, 0.5 M NaPO₄, 1 mM EDTA at 50° C. with washing in 0.5× SSC, 0.1% SDS at 50° C.; and more preferably in 7% SDS, 0.5 M NaPO₄, 1 mM EDTA at 50° C. with washing in 0.1× SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

The terms to “treat” or “treatment,” as used herein, refer to administering rabies virus-neutralizing antibodies or compounds to reduce the frequency with which the effects or symptoms of a rabies virus infection are experienced, to reduce the severity of symptoms, or to prevent effects or symptoms from occurring.

### DETAILED DESCRIPTION OF THE INVENTION

In the practice of the present invention, a rabies virus infection is treated or prevented by administering recombinant rabies virus-neutralizing human antibodies to a subject in need of such treatment. The recombinant rabies virus-neutralizing human antibodies bind specifically to the glycoprotein of various rabies virus strains. Postexposure treatment with two or more different antibodies can neutralize a rabies virus at the site of entry and can prevent the virus from spreading to the central nervous system (CNS). Because viral replication is restricted almost exclusively to neuronal cells, neutralization and clearance of the virus by the antibodies of the present invention prior to entry into the CNS is an effective post-exposure prophylactic.

In the practice of the invention, a rabies virus-associated disorder is treated by a postexposure prophylactic treatment with a mixture of recombinant rabies virus-neutralizing human antibodies. This treatment attains the level of safety, as well as replicates the protective activities, of HRIG.

In one embodiment, two or more different recombinant rabies virus-neutralizing human antibodies are administered to a subject. The recombinant rabies virus-neutralizing human antibodies need not be administered in a single composition. For example, each different recombinant rabies virus-neutralizing human antibody can be administered in a separate composition, or the recombinant rabies virus-neutralizing human antibodies can be administered together in compositions comprising two or more recombinant rabies virus-neutralizing human antibodies. In another aspect, three or more different recombinant rabies virus-neutralizing human antibodies are delivered to the subject.

In one embodiment of the invention, rabies virus infection, or the development of clinical symptoms of rabies, is prevented in subjects who have been exposed to rabies virus by administering at least two different recombinant rabies virus-neutralizing human antibodies. In another embodiment of the invention, rabies virus infection is prevented in subjects who are at risk of exposure to rabies virus by administering at least two different recombinant rabies virus-neutralizing human antibodies.

A mixture of human antibodies selected for administration preferably should: 1) be of IgG isotypes; 2) neutralize more than one rabies virus strain, preferably several, and other lyssaviruses as well; and 3) differ in their epitope-recognition specificities, to prevent the escape of neutralization-resistant variants, (WHO consultation on a monoclonal antibody cocktail for rabies post exposure treatment, WHO, 23-24 May 2002).

In one embodiment of the invention, the recombinant rabies virus-neutralizing human antibodies are derived from monoclonal antibodies of the hybridomas, JA, JB, and J57. The recombinant rabies virus-neutralizing human antibodies are designated “SOJA,” “SOJB,” and “SO57,” respectively. Each of the human antibodies neutralizes more than one strain of rabies virus: 1) human monoclonal antibody SO57 neutralizes fixed (e.g., Pitman-Moore, challenge-virus standard [CVS], and Evelyn-Rokitnicki-Abelseth) and street (e.g., dog RV 4 [DRV-4] and silver-haired-bat rabies virus 18 [SHBRV-18]) RV strains (Dietzschold et al., J. Virol. 1990 64:3087-90); 2) human monoclonal antibody SOJB neutralizes European bat virus 2 (EBV-2); and 3) human monoclonal antibody SOJA neutralizes EBV-2, Lagos bat virus, and Mokola viruses (Champion et al., J. Immunol. Methods 2000 235:81-90; Hanlon et al., Vaccine 2001 19:3834-42). This indicates that the three human monoclonal antibodies recognize different epitopes.

The monoclonal antibody SOJA comprises a light chain comprising the amino acid sequence SEQ ID NO:2 (GenBank Accession No. AAO17825) and a heavy chain comprising the amino acid sequence SEQ ID NO:1 (GenBank Accession No. AA017823). The monoclonal antibody SOJB comprises a light chain comprising the amino acid sequence SEQ ID NO:6 (GenBank Accession No. AAO17826) and a heavy chain comprising the amino acid sequence SEQ ID NO:4 (GenBank Accession No. AAO17822). The monoclonal antibody SO57 comprises a light chain comprising the amino acid sequence SEQ ID NO:7 (GenBank Accession No. AAO17824) and a heavy chain comprising the amino acid sequence SEQ ID NO:9 (GenBank Accession No. AAO17821).

The light chain of the monoclonal antibody SOJA is encoded by a nucleic acid comprising the nucleic acid sequence SEQ ID NO:10 (GenBank Accession No. AY172961) and the heavy chain is encoded by a nucleic acid comprising the nucleic acid sequence SEQ ID NO:11 (GenBank Accession No. AY172959). The light chain of the monoclonal antibody SOJB is encoded by a nucleic acid comprising the nucleic acid sequence SEQ ID NO:5 (GenBank Accession No. AY172962) and the heavy chain is encoded by a nucleic acid comprising the nucleic acid sequence SEQ ID NO:3 (GenBank Accession No. AY172958). The light chain of the monoclonal antibody SO57 is encoded by a nucleic acid comprising the nucleic acid sequence SEQ ID NO:12 (GenBank Accession No. AY172960) and the SO57 heavy chain is encoded by a nucleic acid comprising the nucleic acid sequence SEQ ID NO:8 (GenBank Accession No. AY172957).

In one embodiment of the invention, the recombinant rabies virus-neutralizing human antibody light chains or antibody heavy chains which are used are antibody light chains or antibody heavy chains which have substantial sequence homology or identity with the amino acid sequences of reference antibody light chains or antibody heavy chains. The reference amino acid sequences are SEQ ID NOS:1, 2, 4, 6, 7, and 9. A substantially homologous amino acid sequence refers to a peptide or a portion of a peptide which has an amino acid sequence identity or similarity to a reference peptide of at least about 95%, 96%, 97%, 98%, 99%, or more.

More than one nucleic acid sequence is capable of encoding a particular amino acid sequence. Thus, more than one nucleic acid sequence can encode an antibody light chain and more than one nucleic acid sequence can encode an antibody heavy chain. Degenerate sequences are degenerate within the meaning of the genetic code in that nucleotides can be replaced by other nucleotides in some instances without resulting in a change of the amino acid sequence originally encoded.

In a preferred embodiment, the different recombinant rabies virus-neutralizing human antibodies which are administered are SOJA, SOJB, and SO57. Recombinant SOJA and SO57 IgG1 antibodies are IgG1 antibodies, while SOJB is an IgG3 antibody.

According to some embodiments, the recombinant antibody is a single-chain antibody wherein the heavy chain variable domain and the light chain variable domain are linked by way of a spacer group, preferably a peptide. The single-chain recombinant antibody may further comprise an effector molecule and/or signal sequences facilitating the processing of the antibody by the host cell in which it is prepared.

In one embodiment, the recombinant rabies virus-neutralizing human antibodies are prepared by recombinant DNA techniques comprising culturing transformed host cells under conditions which allow expression of the recombinant antibodies, and then isolating the antibodies.

In one aspect, recombinant rabies virus-neutralizing human antibodies are produced by culturing a host cell which has been transformed with a hybrid vector comprising an expression cassette. The expression cassette comprises a promoter and a nucleic acid sequence encoding the recombinant antibody. In another aspect, the promoter is linked to a first nucleic acid sequence encoding a signal peptide linked in the proper reading frame to a second nucleic acid sequence encoding a recombinant antibody. Expression is controlled by the promoter. The recombinant rabies virus-neutralizing human antibody is then isolated.

Relatively pure antibody preparations are obtained from in vitro production techniques, which allow production to be scaled-up to yield large amounts of the desired antibodies. Techniques for bacterial cell, yeast cell, or mammalian cell cultivation are known in the art and include homogeneous suspension culture, e.g., in an airlift reactor or in a continuous stirrer reactor, or immobilized or entrapped cell culture, e.g. in hollow fibres, microcapsules, on agarose microbeads, or ceramic cartridges.

Antibodies can be prepared by other techniques known to those of skill in the art, and include for example, standard recombinant nucleic acid techniques and chemical synthetic techniques.

The purified antibodies may then be assayed for biological activity according to the assay methods described in the Examples, as well as by methods known to those of skill in the art.

In one embodiment, a hybrid vector comprising a nucleic acid, further comprising a nucleic acid sequence encoding a recombinant rabies virus-neutralizing human antibody, can be used to produce the antibody. Optionally, the hybrid vector comprises an origin of replication or an autonomously replicating sequence, one or more dominant marker sequences, expression control sequences, signal sequences and additional restriction sites.

A recombinant expression vector system encoding an antibody light chain, an antibody heavy chain, or both, can perform two functions in collaboration with compatible host cells. One function is to facilitate the cloning of a nucleic acid that encodes the antibody chain or chains, i.e. to produce usable quantities of nucleic acid (cloning vectors). The other function is to provide for replication and expression of the recombinant gene constructs in a suitable host, either by maintenance as an extrachromosomal element or by integration into the host chromosome (expression vectors). A cloning vector comprises the recombinant nucleic acid constructs as described above, an origin of replication or an autonomously replicating sequence, dominant marker sequences and, optionally, signal sequences and additional restriction sites. An expression vector additionally comprises expression control sequences essential for the transcription and translation of the recombinant nucleic acids, thereby producing a recombinant rabies virus-neutralizing human antibody.

Using recombinant technology, a rabies virus-based vector, which expresses high levels (approximately 60 pg/cell) of rabies virus-neutralizing human antibody can be constructed (Morimoto et al., J. Neurovirol. 6:373-81 2000; Morimoto et al., J. Immunol. Methods 252:199-206 2001; Schnell et al., Proc. Natl. Acad. Sci. USA 97:3544-3549 2000).

In one embodiment, recombinant human antibodies are produced with a Rhabdovirus (RhV) expression system, prepared from a vaccine strain rabies virus-based vector expression system. A RhV expression system for producing rabies virus-neutralizing human antibodies has the following features: 1) because the RhV expression system of the invention is not cytopathic, infected cells can continuously produce antibodies for a long time (>2 weeks), a situation that results in cost-efficient monoclonal antibody production; 2) a variety of mammalian cell cultures are susceptible to infection with the recombinant RhV expression system, and, therefore, cell lines that already have been approved for vaccine or antibody production (e.g., Vero African green monkey cells and CHO cells) can be used for monoclonal antibody production by RhV; and 3) RhV can easily be destroyed by use of either UV radiation or chemicals (e.g., nonionic detergents or ethanol) that do not alter the activity of the antibody. Moreover, the RhV can be modified to contain a vesicularstomatitisvirus glycoprotein (G) protein gene, instead of the rabies virus G gene that carries the major determinants responsible for the pathogenicity of RV (Schnell et al., Proc. Natl. Acad. Sci. USA 2000 97:3544-49; Dietzschold et al., Proc. Natl. Acad. Sci. USA 1983 80:70-4). Therefore, a recombinant rhabdovirus expression system for producing rabies virus-neutralizing human antibodies presents limited biosafety concerns.

Both heavy and light chain antibody sequences encoding the heavy and light chain peptides of a human antibody can be inserted into a recombinant rhabdovirus expression vector at the site modified between the glycoprotein gene and the polymerase gene (Schnell et al., Proc. Natl. Acad. Sci. USA 97:3544-3549 2000). Furthermore, this recombinant rhabdovirus expression system can infect a variety of mammalian cell lines and is non-cytolytic, allowing the use of cell culture technology routinely employed to produce rabies vaccines (Morimoto et al., J. Neurovirol. 6:373-81 2000; Morimoto et al., J. Immunol. Methods 252:199-206 2001; Schnell et al., Proc. Natl. Acad. Sci. USA 97:3544-3549 2000).

In one embodiment, an origin of replication or an autonomously replicating sequence is provided either by construction of the vector to include an exogenous origin such as derived from Simian virus 40 (SV 40) or another viral source, or by the host cell chromosomal mechanisms.

In another embodiment, selection markers in a vector allow for selection of host cells which contain the vector. Selection markers include genes which confer resistance to heavy metals such as copper or to antibiotics such as geneticin (G-418) or hygromycin, or genes which complement a genetic lesion of the host cell such as the absence of thymidine kinase, hypoxanthine phosphoryl transferase, dihydrofolate reductase or the like.

In some embodiments, secretion of recombinant antibodies is directed. Signal sequences may be, for example, presequences or secretory leaders directing the secretion of the recombinant antibody, splice signals, or the like. Examples for signal sequences directing the secretion of the recombinant antibody are sequences derived from the ompA gene, the pelB (pectate lyase) gene, or the phoA gene.

Transcription of antibody sequences is regulated by a promoter and by sequences necessary for the initiation and termination of transcription and for stabilizing the mRNA and, optionally, enhancers and further regulatory sequences. A wide variety of promoter sequences may be employed, depending on the nature of the host cell. Promoters that are strong and at the same time well regulated are the most useful. Sequences for the initiation of translation are for example Shine-Dalgarno sequences. Sequences necessary for the initiation and termination of transcription and for stabilizing the mRNA are commonly available from the noncoding 5′-regions and 3′-regions, respectively, of viral or eukaryotic cDNAs, e.g. from the expression host. Enhancers are transcription-stimulating DNA sequences of viral origin, e.g. derived from Simian virus, polyoma virus, bovine papilloma virus or Moloney sarcoma virus, or of genomic, especially murine, origin.

The various nucleic acid segments of the vector are operationally linked, i.e., they are contiguous and placed into a functional relationship with each other. Examples of vectors which are suitable for replication and expression in an E. coli strain are bacteriophages, for example derivatives of λ bacteriophages, or plasmids, such as, in particular, the plasmid ColE1 and its derivatives, for example pMB9, pSF2124, pBR317 or pBR322 and plasmids derived from pBR322, such as pUC9, pUCK0, pHRi148 and pLc24. Suitable vectors contain a complete replicon, a marker gene, recognition sequences for restriction endonucleases, so that the foreign DNA and, if appropriate, the expression control sequence can be inserted at these sites, and optionally signal sequences and enhancers.

Microbial promoters are, for example, the strong leftward promoter PL of bacteriophage λ. which is controlled by a temperature sensitive repressor. Also suitable are E. coli promoters such as the lac (lactose) promoter regulated by the lac repressor and induced by isopropyl-β-D-thiogalactoside, the trp (tryptophan) promoter regulated by the trp repressor and induced e.g. by tryptophan starvation, and the tac (hybrid trp-lac promoter) regulated by the lac repressor.

A variety of vectors are also suitable for replication and expression in yeast and contain a yeast replication start site and a selective genetic marker for yeast. One group of such vectors includes so-called ars sequences (autonomous replication sequences) as origins of replication. These vectors are retained extrachromosomally within the yeast cell after transformation and are replicated autonomously. Furthermore, vectors which contain all or part of the 2 μm plasmid DNA from Saccharomyces cerevisiae can be used. Such vectors will get integrated by recombination into 2 μm plasmids already existing within the cell, or replicate autonomously. 2 μm sequences are particularly suitable when high transformation frequency and high copy numbers are to be achieved.

Expression control sequences which are suitable for expression in yeast are, for example, those of highly expressed yeast genes. Thus, the promoters for the TRP1 gene, the ADHI or ADHII gene, acid phosphatase (PHO3 or PHO5) gene, isocytochrome gene or a promoter involved with the glycolytic pathway, such as the promoter of the enolase, glyceraldehyde-3-phosphate kinase (PGK), hexokinase, pyruvate decarboxylase, phosphofructokinase, glucose-6-phosphate isomerase, 3-phosphoglycerate mutase, pyruvate kinase, triosephosphate isomerase, phosphoglucose isomerase and glucokinase genes, can be used.

Vectors suitable for replication and expression in mammalian cells are preferably provided with promoting sequences derived from DNA of viral origin, e.g. from Simian virus 40 (SV40), Rous sarcoma virus (RSV), adenovirus 2, bovine papilloma virus (BPV), papova-virus BK mutant (BKV), rhabdovirus, rabies virus, or mouse or human cytomegalovirus (CMV). Alternatively, the vectors may comprise promoters from mammalian expression products, such as actin, collagen, myosin etc., or the native promoter and control sequences which are normally associated with the desired gene sequence, i.e. the immunoglobulin H-chain or L-chain promoter.

Other vectors are suitable for both prokaryotic and eukaryotic hosts and are based on viral replication systems. Particularly preferred are vectors comprising Simian virus promoters, e.g. pSVgpt or pSVneo, further comprising an enhancer, e.g. an enhancer normally associated with the immunoglobulin gene sequences, in particular the mouse Ig H- or L-chain enhancer.

Different approaches can be followed to obtain complete tetrameric light chain and heavy chain antibodies. In one embodiment, antibody light chains and antibody heavy chains are co-expressed in the same cells to achieve intracellular association and linkage of antibody light chains with antibody heavy chains into complete tetrameric light chain and heavy chain antibodies.

In one embodiment, a nucleic acid encoding an antibody light chain and a nucleic acid encoding an antibody heavy chain are present on two mutually compatible expression vectors which are each under the control of different or the same promoter(s). In this embodiment, the expression vectors are co-transformed or transformed individually.

In one embodiment of the invention, host cells are transformed with a recombinant rhabdovirus expression system comprising nucleic acid sequences encoding an antibody light chain and/or an antibody heavy chain of the desired recombinant antibody. Thus, in one aspect, a nucleic acid sequence can encode a single-chain recombinant antibody.

Examples of suitable hosts are bacteria, in particular strains of Escherichia coli, for example E. coli X1776, E. coli Y1090, E. coli HB 101, E. coli W3110, E. coli HB 101/LM1035, E. coli JA 221, E. coli DH5α, E. coli K12, or E. coli CC118 strain, Bacillus subtilis, Bacillus stearothermophilus, Pseudomonas, Haemophilus, Streptococcus and others, and yeasts, for example Saccharomyces cerevisiae such as S. cerevisiae GRF 18. Further suitable host cells are cells of higher organisms, in particular established continuous human or animal cell lines, e.g. human embryonic lung fibroblasts L132, human malignant melanoma Bowes cells, HeLa cells, SV40 virus transformed kidney cells of African green monkey COS-7, BHK cells, BSR cells, VERO cells, or Chinese hamster ovary (CHO) cells, or cells of lymphoid origin, such as lymphoma, myeloma, hybridoma, trioma or quadroma cells, for example PAI, Sp2/0 or X63-Ag8. 653 cells.

In one embodiment, transformed host cells are prepared wherein suitable recipient host cells are transformed with a hybrid vector, and the transformed cells are selected using criteria known in the art. Transformation of microorganisms is carried out as described in the literature, for example for S. cerevisiae (A. Hinnen et al., Proc. Natl. Acad. Sci. USA 75:1929 1978), for B. subtilis (Anagnostopoulos et al., J. Bacteriol. 81:741 1961), and for E. coli (M. Mandel et al., J. Mol. Biol. 53:159 1970).

Accordingly, the transformation procedure of E. coli cells includes, for example, Ca²⁺ pretreatment of the cells so as to allow DNA uptake, and incubation with the hybrid vector. The subsequent selection of the transformed cells can be achieved, for example, by transferring the cells to a selective growth medium which allows separation of the transformed cells from the parent cells dependent on the nature of the marker sequence of the vector DNA. Preferably, a growth medium is used which does not allow growth of cells which do not contain the vector. The transformation of yeast comprises, for example, steps of enzymatic removal of the yeast cell wall by means of glucosidases, treatment of the obtained spheroplasts with the vector in the presence of polyethylene glycol and Ca²⁺ ions, and regeneration of the cell wall by embedding the spheroplasts into agar. Preferably, the regeneration agar is prepared in a way to allow regeneration and selection of the transformed cells as described above at the same time.

Transformation of cells of higher eukaryotic origin, such as mammalian cell lines, is achieved by methods such as infection or transfection. Transfection is carried out by conventional techniques, such as calcium phosphate precipitation, microinjection, protoplast fusion, electroporation, i.e. introduction of DNA by a short electrical pulse which transiently increases the permeability of the cell membrane, or in the presence of helper compounds such as diethylaminoethyldextran, dimethyl sulfoxide, glycerol or polyethylene glycol, and the like. After the transfection procedure, transfected cells are identified and selected, for example, by cultivation in a selective medium chosen depending on the nature of the selection marker, for example standard culture media such as Dulbecco's modified Eagle medium (DMEM), minimum essential medium, RPMI 1640 medium and the like, containing e.g. the corresponding antibiotic.

In a preferred embodiment, the recombinant rhabdovirus expression system comprising a nucleic acid comprising a nucleic acid sequence encoding an antibody light chain or antibody heavy chain, or both, is used to infect mammalian cells such as BSR cells, CHO cells, VERO cells, and BHK cells, or other cells approved for antibody production. The cells can be cultured under conditions which allow production of the antibody of the invention. The antibody can be isolated and purified by techniques known in the art.

Antibody light chains or antibody heavy chains of the recombinant rabies virus-neutralizing human antibodies to be administered can be modified with other substances. Methods of modifying the antibodies with other substances, in particular labels, are well known to those skilled in the art. Modification of the antibodies may alter their activity, for example by altering characteristics such as in vivo tissue partitioning, peptide degradation rate, or rabies virus-neutralizing activity. The modifications may also confer additional characteristics to the compound, such as the ability to be detected, manipulated, or targeted.

The recombinant rabies virus-neutralizing human antibodies can be modified with polymeric and macromolecular structures (e.g., liposomes, zeolites, dendrimers, magnetic particles, and metallic beads) or targeting groups (e.g., signal peptide sequences, ligands, lectins, or antibodies). The modifying substance may be joined to a chain, for example, by chemical means (e.g., by covalent bond, electrostatic interaction, Van der Waals forces, hydrogen bond, ionic bond, chelation, and the like) or by physical entrapment. For example, the antibodies may be modified with a label (e.g., substances which are magnetic resonance active; radiodense; fluorescent; radioactive; detectable by ultrasound; detectable by visible, infrared or ultraviolet light). Suitable labels include, for example, fluorescein isothiocyanate, peptide chromophores such as phycoerythrin or phycocyanin and the like; bioluminescent peptides such as the luciferases originating from Photinus pyrali; or fluorescent proteins originating from Renilla reniformi.

In one aspect of the invention, the antibodies may contain aspartic acid (D) residues to promote their solubility. In another aspect, antibody longevity is enhanced by the addition of adducts such as sucrose or polyethylene glycol.

One skilled in the art can readily determine an effective amount of recombinant rabies virus-neutralizing human antibodies to be administered to a given subject, by taking into account factors such as the size and weight of the subject; the extent of disease penetration; the age, health and sex of the subject; the route of administration; and whether the administration is regional or systemic. Generally, the amount of antibody administered to a subject depends upon the amount of rabies virus that needs to be neutralized and the amount of rabies virus-neutralizing activity exhibited by the antibodies. Those skilled in the art may derive appropriate dosages and schedules of administration to suit the specific circumstances and needs of the subject. For example, suitable doses of each antibody to be co-administered can be estimated from the amount of rabies virus to which a subject has been exposed, or the amount of rabies virus to which the subject is in risk of being exposed. Typically, dosages of antibody are between about 0.001 mg/kg and about 100 mg/kg body weight. In some embodiments, dosages are between about 0.01 mg/kg and about 60 mg/kg body weight.

It is understood that the effective dosage will depend on the age, sex, health, and weight of the recipient, kind of concurrent treatment, if any, frequency of treatment, and the nature of the effect desired. The most preferred dosage will be tailored to the individual subject, as is understood and determinable by one of skill in the art, without undue experimentation.

A mixture of recombinant rabies virus-neutralizing human antibodies can be administered in equimolar concentrations to a subject in need of such treatment. In another instance, the antibodies are administered in concentrations which are not equimolar. In other instances, the antibodies are administered as equal amounts of protein, by weight, per kilogram of body weight. For example, the antibodies can be administered in equal amounts, based on the weight of the subject. In another instance, the antibodies are administered in unequal amounts. In yet other instances, the amount of each antibody to be administered is based on its neutralizing activity. For example, a mixture with between about 1 IU/kg body weight and about 50 IU/kg body weight of rabies virus-neutralizing activity can be administered.

In general, the schedule or timing of administration of a mixture of rabies virus-neutralizing human antibodies is according to the accepted practice for the procedure being performed.

When used in vivo, the antibodies are preferably administered as a pharmaceutical composition, comprising a mixture, and a pharmaceutically acceptable carrier. The antibodies may be present in a pharmaceutical composition in an amount from 0.001 to 99.9 wt %, more preferably from about 0.01 to 99.0 wt %, and even more preferably from 0.1 to 50 wt %. To achieve good plasma concentrations, an antibody, or a combination of antibodies, may be administered, for example, by intravenous injection, as a solution comprising 0.1 to 1.0% of the active agent.

All of the different recombinant rabies virus-neutralizing human antibodies to be administered need not be administered together in a single composition. The different recombinant rabies virus-neutralizing human antibody can be administered in separate compositions. For example, if three different antibodies are to be administered, the three different antibodies can be delivered in three separate compositions. In addition, each antibody can be delivered at the same time, or the antibodies can be delivered consecutively with respect to one another. Thus, the mixture of recombinant rabies virus-neutralizing human antibodies can be administered in a single composition, or in multiple compositions comprising one or more recombinant rabies virus-neutralizing human antibodies.

The recombinant rabies virus-neutralizing human antibodies, or pharmaceutical compositions comprising these compounds, may be administered by any method designed to allow compounds to have a physiological effect. Administration may occur enterally or parenterally; for example orally, rectally, intracisternally, intravaginally, intraperitoneally, locally (e.g., with powders, ointments or drops). Parenteral administration is preferred. Particularly preferred parenteral administration methods include intravascular administration (e.g., intravenous bolus injection, intravenous infusion, intra-arterial bolus injection, intra-arterial infusion and catheter instillation into the vasculature), peri- and intra-target tissue injection, subcutaneous injection or deposition including subcutaneous infusion (such as by osmotic pumps), intramuscular injection, intraperitoneal injection, and direct application to the target area, for example by a catheter or other placement device.

Pharmaceutically acceptable carriers include physiologically tolerable or acceptable diluents, excipients, solvents, or adjuvants. The compositions are preferably sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g. 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions can be prepared in conventional forms, either as liquid solutions or suspensions, solid forms suitable for solution or suspension in liquid prior to injection, or as emulsions. Compositions for intramuscular, intraperitoneal, subcutaneous, or intravenous application are e.g. isotonic aqueous solutions or suspensions, optionally prepared shortly before use from lyophilized or concentrated preparations: Suspensions in oil contain as oily component the vegetable, synthetic or semi-synthetic oils customary for injection purposes. The pharmaceutical compositions may be sterilized and contain adjuncts, e.g. for conserving, stabilizing, wetting, emulsifying or solubilizing the ingredients, salts for the regulation of the osmotic pressure, buffer and/or compounds regulating the viscosity, e.g. sodium carboxycellulose, carboxymethylcellulose, sodium carboxymethylcellulose, dextran, polyvinylpyrrolidine or gelatin. Pharmaceutical compositions according to the present invention can be prepared in a manner fully within the skill of the art.

Where the administration of the antibody is by injection or direct application, the injection or direct application may be in a single dose or in multiple doses. Where the administration of the compound is by infusion, the infusion may be a single sustained dose over a prolonged period of time or multiple infusions.

The invention should not be construed to be limited solely to the assays and methods described herein, but should be construed to include other methods and assays as well. One of skill in the art will know that other assays and methods are available to perform the procedures described herein.

Without further description, it is believed that one of ordinary skill in the art can, using the preceding description and the following illustrative examples, make and utilize recombinant rabies virus-neutralizing human antibodies and practice the claimed methods. The following working examples therefore, specifically point out the preferred embodiments of the present invention, and are not to be construed as limiting in any way the remainder of the disclosure.

### EXAMPLE 1

### Production and Virus-Neutralizing Activity of Anti-Rabies-Virus Recombinant-Expressed Human Antibodies (rhuMAbs)

Preparation of Antibody cDNAs.

Three hybridomas, JA, JB, and J57, secrete the human monoclonal antibodies designated as “SOJA,” “SOJB,” and “SO57.” Details of the generation and analysis of these monoclonal antibodies have been reported elsewhere (Dietzschold et al., J. Virol. 1990 64:3087-90; Champion et al., J. Immunol. Methods 2000 235:81-90). Immunoglobulin heavy chain (Ig H) and immunoglobulin light chain (Ig L) mRNAs were isolated from the JA, JB, and J57 hybridomas, and a rhabdovirus vector (RhV) was used to express the antibodies at high levels in BSR or CHO cells.

To obtain the complete nucleic-acid sequences of the Ig H and Ig L mRNAs, total RNA was isolated from each hybridoma cell line using the Tri-Reagent protocol (Sigma) and the RNeasy RNA extraction kit (Qiagen), according to the manufacturer's recommendations.

The Ig L and Ig H cDNA fragments were amplified using the Rapid Amplification of cDNA Ends kit (GIBCO-BRL) and gene-specific primers (GSPs) corresponding to the 3′ ends of the constant-region Ig H and Ig L genes. In brief, 2.5 μg of total RNA was reverse transcribed using a ThermoScript reverse transcriptase (Life Technologies) and a GSP, for 60 min at 55° C. The mRNA was then degraded by RNase H, and the cDNA was purified using GlassMAX spin cartridges. After dC-tailing of the cDNA by terminal deoxynucleotidyltransferase and dCTP, the cDNA was PCR-amplified using the Abridged Anchor Primer (GIBCO-BRL) and a second GSP, to obtain a nested product. The PCR products were reamplified using a third GSP, were purified by gel electrophoresis, and were sequenced. As a final step, sequences were analyzed by both DNASTAR software (DNAstar) and National Center for Biotechnology Information website tools.

To clone full-length Ig H and Ig L cDNAs, 1 μg of total RNA from each hybridoma was reverse transcribed using 10 pmol of oligo dT primer, 200 units of Superscript II, and 20 units of RNase inhibitor, in a 40-μl reaction, with First Strand Buffer, 200 μM dNTP, and 10 mM dithiothreitol. A 5-μl portion of the reverse-transcription reaction was then PCR-amplified using the Expand High Fidelity PCR System (Roche) and 10 pmol of primers complementary to the 5′ end and the 3′ end of the Ig L and Ig H cDNAs.

The cloned Ig cDNAs were sequenced and the GenBank accession numbers are as follows: SOJA Ig L, AY172961 (SEQ ID NO:10); SOJA Ig H, AY172959 (SEQ ID NO:11); SOJB Ig L, AY172962 (SEQ ID NO:5); SOJB Ig H, AY172958 (SEQ ID NO:3); SO57 Ig L, AY172960 (SEQ ID NO:12); and SO57 Ig H, AY172957 (SEQ ID NO:8). For gene assembly, JA, JB, and J57 Ig H cDNAs were reamplified by use of a primer corresponding to the 5′ end of the cDNAs (JAHF, 5′-AAACGTACGATGGAGTTTGGGCTGAGCTGGCTT-3′ (SEQ ID NO:13); JBHF, 5′-AACGTACGATGGACACACTTTGCTCCACGCTCCT-3′ (SEQ ID NO:14); and J57HF, 5′-AAACGTACGACCATGGACTGGACCTGGAGGTTCCT-3′ (SEQ ID NO:15)) and HR primer 5′-TGCTAGGGGTGTTAGTTTTTTTCATGACTCATTTACCCGGGGACAGGG A-3′ (SEQ ID NO:16), which is complementary to the 3′ end of each Ig H cDNA and to a linker sequence consisting of rabies-virus transcription stop/start signal (Morimoto et al., J. Immunol. Methods 2001 252:199-206).

The Ig L cDNAs were reamplified using LF primers, including a linker region and regions specific to the 5′ end of the different light-chain cDNAs (5′-GGTAAATGAGTCATGAAAAAAACTAACACCCCTAGCNNNNNNNNN NNNNNNNNNN-3′ (SEQ ID NO:17), where N is a 5′ end of the light chain of cDNAs) and a primer for the 3′ end of cDNAs (JALR, 5′-AAAGCTAGCCTAACACTC-TCCCCTGTTGAAGCTC-3′ (SEQ ID NO: 18); JBLR, 5′-AAAGCTAGCCTATGAACATTCTGTAGGGGCCACTGT-3′ (SEQ ID NO:19); and J57LR, 5′-AAATCTAGACTATGAACATTCTGTAGGGGCCAC-3′ (SEQ ID NO:20)).

Construction of a Recombinant Rhabdovirus Vector.

A recombinant rhabdovirus vector (RhV) was prepared starting with a rabies virus vector described previously (Foley et al., Proc. Natl. Acad. Sci. USA 2000 97:14680-14685). The ecto- and transmembrane domains of a rabies virus (GenBank Accession No. NC_{—}001542) glycoprotein gene (GenBank Accession No. NP_{—}056796) were replaced with the ecto- and transmembrane domains of a vesicularstomatitisvirus glycoprotein gene CVSV G) (GenBank Accession No. J02428) (Foley et al., Proc. Natl. Acad. Sci. USA 2000 97:14680-14685; Schnell et al., J. Virology 1996 70:2318-2323; Lawson et al., Proc. Natl. Acad. Sci. USA 1995 92:4477-4481), yielding a chimeric glycoprotein gene in the vector. The resulting plasmid was named pSN-VSV-G.

The rabies virus glycoprotein gene carries the major determinants responsible for the pathogenicity of rabies virus (Dietzschold et al., Proc. Natl. Acad. Sci. USA 1983 80:70-74). By replacing the rabies virus glycoprotein gene ecto- and transmembrane sequences with vesicularstomatitisvirus glycoprotein gene ecto- and transmembrane sequences, a recombinant rhabdovirus expression system is formed which has limited biosafety concerns.

To construct the pSN-VSV-G vector, the cytoplasmic tail of rabies virus G was PCR amplified from pSN with the primers RP8,5′-CCTCTAGATTACAGTCTGGTCTCACCCCC-3′ (XbaI, bold) (SEQ ID NO:21) and RP29, 5′-CCCGGGTTAACAGAAGAGTCAATCGATCAGAAC-3′ (HpaI, bold) (SEQ ID NO:22) (Foley et al., Proc. Natl. Acad. Sci. USA 2000 97:14680-14685). The ecto- and transmembrane domains of the VSV G gene (GenBank Accession No. J02428) were amplified from pVSV-XN1 (Schnell et al., J. Virology 1996 70:2318-2323) with the primers RP33, 5′-TTAAGTTAACCAAGAATAGTCCAATGA-3′ (HpaI, bold) (SEQ ID NO:23) and RP34, 5′-TCTCGAGCCCGGGACTATGAAGTGCCTTTTGTAC-3′ (XbaI, bold) (SEQ ID NO:24). Both PCR products were digested with HpaI and ligated. The ligation products were PCR reamplified with the primers RP8 and RP34, and the PCR product was cloned into the XmaI and XbaI sites of pSN. The resulting plasmid was designated pSN-VSV-G (Foley et al., Proc. Natl. Acad. Sci. USA 2000 97:14680-1468).

The plasmid pSN-VSV-G described above (hereinafter referred to as pSPBN) was further modified to allow insertion and expression of nucleic acids comprising sequences encoding antibody light chain sequences, heavy chain sequences, or both. The plasmid was modified by introducing BsiWI and NheI sites between the glycoprotein (G) gene and the polymerase (L) gene using a PCR strategy (see for example, Foley et al., Proc. Natl. Acad. Sci. USA 2000 97:14680-1468 and Schnell et al., Proc. Natl. Acad. Sci. USA 2000 97:3544-3549). The resulting recombinant rhabdovirus vector is referred to as RhV.

Preparation of Plasmids and Recombinant Viruses Comprising Light and Heavy Chain Nucleic Acid Sequences.

The Ig H and Ig L cDNAs prepared above were linked by PCR, and the resulting Ig L+linker+Ig H cDNA was digested by BsiWI and NheI and was inserted into the corresponding sites of plasmid pSPNB described above. Three different Ig L+Ig H cDNAs were inserted, which resulted in plasmids pSPBN-SOJA, pSPBN-SOJB, and pSPBN-SO57.

Production of Antibodies by Mammalian Cells.

The recombinant viruses generated from the above plasmids were rescued as described elsewhere (Morimoto et al., J. Neurovirol. 2000 6:373-81) and were used to infect either BSR cells, which is a subclone of baby hamster kidney (BHK) cells, or to infect CHO cells, at an MOI of 0.1. After infection, the cells were incubated with Cellgro-FREE culture medium (Mediatech) at 34° C. After 3 days of incubation, tissue-culture supernatants were collected and were subjected to UV irradiation to inactivate the virus.

Table 1 demonstrates the amounts of three different recombinant-expressed human antibodies that were secreted into the tissue culture supernatant by infected BSR cells. It can be seen that the BSR cells infected with recombinant viruses comprising nucleic acids encoding the SOJA, SOJB, or SO57 antibodies each produced recombinant human antibody at a rate of about 40 μg/ml/48 hours. Infection of either BSR cells or CHO cells yielded high level production (<40 μg/mL/48 hours).

TABLE 1
Production and virus-neutralizing activity of anti-rabies-virus
recombinant-expressed human monoclonal antibodies (rhuMAbs).

| | Production | | | | |
|---|---|---|---|---|---|
| | in BSR | Challenge virus | | | |
| Antibody | cells, | CVS- | | | |
| (isotype) | μg/mL/48 h | 11 | CVS-N2c | SHBRV-18 | DRV-4 |
| SOJA (IgG1)^{a} | 40 | 30 | 120 | 60 | 40 |
| SOJB (IgG3)^{a} | 37 | 240 | 360 | 60 | 540 |
| SO57 (IgG1)^{a} | 40 | 2200 | 1620 | 360 | 1080 |
| rhuMAb | — | 720 | 1080 | 180 | 810 |
| mixture^{a} | | | | | |
| HRIG^{b} | — | 110 | 202 | 108 | 108 |

NOTE.
“CVS” is challenge-virus standard;
“DRV-4” is dog rabies virus 4;
“HRIG” is human rabies immunoglobulin;
“SHBRV-18” silver-haired-bat rabies virus.
^{a}Purified rhuMAbs and the rhuMAb mixture (SOJA:SOJB:SO57 protein ratio, 1:1:1) were adjusted, before testing, to a protein concentration of 1 mg/mL.
^{b}HRIG was used as produced by the manufacturer (150 IU/mL; Bayer).

Purification of Antibodies by Affinity Chromatograph.

The individual recombinant human antibodies secreted into the tissue culture medium were then purified from the culture supernatants and each sample was calibrated and adjusted to the same protein content.

IgG1 antibody was purified using a protein A column (rProtein A Sepharose™ Fast Flow, Amersharn Pharmacia Biotech). Briefly, supernatants were clarified by filtration through a 0.45 μm membrane and the pH adjusted to 8.0 with 1N NaOH. Supernatant was run through the column at a linear flow rate of approximately 100 cm/hour. After washing in PBS (pH 8), antibody was eluted from the column using a 0.1M citric acid solution and then dialyzed against PBS.

IgG3 antibody was purified using a protein G column (Protein G Sepharose™ Fast Flow, Amersham Pharmacia Biotech). IgG3-containing supernatant was clarified by filtration through a 0.45 μm membrane and the pH adjusted to 7.0 with 1N NaOH. Supernatant was run through the column at a linear flow rate of approximately 11 cm/hour. After washing with PBS, antibody was eluted from the column using 0.1M glycine buffer, pH 3.0, and then dialyzed against PBS.

Protein concentrations of the dialyzed antibody preparations were determined using a protein detection assay (Bio-Rad Laboratories, Hercules Calif.) as follows. 100 μl of sample were added to 5 ml of a ⅕ dilution of dye reagent concentrate and incubated at room temperature for 10 minutes. A negative PBS control and various bovine serum albumin (BSA) protein standards were included in each assay. After incubation, samples were read in a spectrophotometer at 595 nm. Protein concentrations of test samples were calculated with reference to the absorbance of the BSA standards. The purity of all antibody preparations was assessed by electrophoresis in 12.5% polyacrylamide gel under reducing conditions (SDS-PAGE). Purified antibodies showed two major bands on SDS-PAGE corresponding to isolated heavy and light immunoglobulin chains.

Virus-Neutralizing Activity.

The amount of virus-neutralizing activity produced by the mammalian cells infected with a recombinant rhabdovirus expression system comprising recombinant rabies virus-neutralizing human antibody was determined by the rapid fluorescent focus-inhibition test (RFFIT) (Morimoto et al., J. Immunol. Methods 2001 252:199-206). One method of determining whether a reduction in viral titer is positive, is when a reduction of viral titer of >100 infective units is achieved (Dietzschold et al., J. Virol. 1990 64:3087-3090). In addition, virus-neutralizing antibody (VNA) titers can be expressed in international units with National Institutes of Health reference serum as the standard. Neutralizing activity is typically expressed as units per microgram of protein or as units per milliliter. The rabies virus strains used for virus neutralization are described elsewhere (Dietzschold et al., J. Hum. Virol. 2000 3:50-7; Morimoto et al., Proc. Natl. Acad. Sci. USA 1998 95:3152-6). Supernatant samples from each transformed cell line were assayed for the presence of rabies virus-neutralizing antibodies using the RFFIT. Supernatant samples (50 μl) were diluted in 96 well flat-bottom plates (Nunc). Rabies virus dilution known to cause 80-90% infection of the indicator cells were added to each test sample, and the plates incubated at 37° C. for 1 hour. Negative media and positive rabies-immune serum control samples were included in each assay. After incubation, 30 μl of a 1.8×10⁶ cells/ml concentration of BHK cells were added to each well and cultures incubated overnight at 37° C. The plates were then washed once with ice-cold PBS and fixed with ice-cold 90% acetone for 20 minutes at −20° C. After fixation, acetone was removed and the plates were air dried. To detect infected BHK cells, 40 μl of FITC anti-rabies nucleoprotein monoclonal globulin (Centocor, Malvern Pa.) were added to each well for 45 minutes at 37° C. The plates were then washed three times with distilled water and examined under a fluorescent microscope.

The individual recombinant human antibodies which had been purified from the culture supernatants were tested for their capacity to neutralize fixed (e.g., CVS-N2c and CVS-11) and street (e.g., SHBRV-18 and DRV-4) rabies virus strains. The three recombinant human antibodies exhibit qualitative and quantitative differences in their capacities to neutralize the different rabies viruses, as measured by the rapid fluorescent focus inhibition test (Table 1). Furthermore, the relative ratio of the virus-neutralization antibody (VNA) titers for the rabies virus strains, obtained by a mixture consisting of equal molar concentrations of each of the three recombinant human antibodies, was similar to that obtained with HRIG, except for the anti-SHBRV-18 HRIG VNA titer, which was slightly higher.

### EXAMPLE 2

### Postexposure Prophylaxis of Mice with a Recombinant Anti-Rabies Human Monoclonal Antibody Mixture

To determine the protective activity of the recombinant human antibody mixture of SOJA, SOJB, and SO57 in vivo, female Swiss Webster mice (10 mice/group) were infected intranasally with 10 LD₅₀ of the rabies virus CVS-N2c. One hour later, the mice received a single treatment with the mixture or with HRIG. Each treated group received a mixture comprising different amounts of activity. The mixture was prepared with a SOJA:SOJB:SO57 protein ratio of 1:1:1. The activity of each antibody or a mixture of antibodies is expressed as International units (IU). The IU of the antibodies is determined by titration against a WHO standard antiserum. Mice received the mixture in ranges from 0-20 IU/kg body weight. The mice were then observed for five weeks to determine whether they developed clinical signs of rabies. The effective dose of treatment at which 50% of the animals were protected (ED₅₀) against a lethal challenge was calculated for animals treated with the SOJA:SOJB:SO57 mixture and animals treated with HRIG.

If clinical signs of rabies became evident, the mice were euthanized by CO₂ intoxication. A diagnosis of rabies was confirmed by the direct fluorescent-antibody test performed on impressions of brain tissue from animals suspected to have rabies.

Postexposure prophylactic treatment with the recombinant rabies virus-neutralizing human antibody mixture prevented a lethal rabies virus infection in vivo (Table 2). Furthermore, the protective activity of the human antibody mixture was found to be comparable to the protective activity of HRIG (Table 2). The mixture protected 8 of 10 mice from developing clinical signs of rabies when used at 20 IU/kg, but at a concentration of 2.5 IU/kg, no mice were protected from developing clinical signs of rabies. The ED₅₀ dose for mice developing signs of rabies was 3.38 IU/kg for HRIG and 4.47 Iu/kg for the rhuMAb mixture.

TABLE 2
Postexposure Prophylaxis of Mice With Human Rabies
Immunoglobulin (HRIG) and Anti-Rabies Recombinant-Expressed
Human Monoclonal Antibody (rhuMAb) Mixture.

Antibody

| Concentration^{a} | HRIG | rhuMAb mixture^{b} |
|---|---|---|
| 20 IU/kg | 9/10 | 8/10 |
| 10 IU/kg | 6/10 | 7/10 |
| 5 IU/kg | 6/10 | 4/10 |
| 2.5 IU/kg | 0/10 | 0/10 |
| ED₅₀, IU/kg | 3.38 | 4.47 |

^{a}Amount of HRIG or mixture administered.
^{b}SOJA:SOJB:SO57 protein ratio, 1:1:1.
Data are expressed as number of mice with clinical signs of rabies/number of mice tested.

### EXAMPLE 3

### Postexposure Prophylaxis of Hamsters with a Recombinant Anti-Rabies Human Monoclonal Antibody Mixture

The efficacy of the recombinant human antibody mixture was further examined in a hamster postexposure prophylaxis model.

Two-month-old (100 gram; 10/group) female Syrian hamsters (Harlan-Sprague-Dawley) were challenged with 50 μl of a homogenate of salivary-gland tissue from a naturally infected rabid coyote. Animals were inoculated in the left gastrocnemius muscle. Four hours after inoculation with the rabies virus, postexposure prophylactic treatment consisting of a rhuMAb mixture (as described in Example 2) at 20 IU/kg was initiated in 10 hamsters. Fifty microliters of the antibody preparation was administered once, at the same site where the animal was inoculated with virus. Ten hamsters that did not receive the postexposure prophylactic treatment mixture served as control animals.

Thereafter, animals were observed daily for 90 days. If clinical signs of rabies became evident in an animal, it was euthanized by CO₂ intoxication. A diagnosis of rabies was confirmed following euthanasia. A diagnosis of rabies was confirmed using the direct fluorescent-antibody test, which was performed on impressions of brain tissue from animals suspected of having rabies.

All 10 hamsters administered the antibody mixture of SOJA/SOJB/SO57 survived. However, all of the control animals died from rabies. The dose of virus used contained a ˜10^{6.8} MIC LD₅₀/mL concentration of a canine RV variant (COSRV) that is in circulation in the United States/Mexico border area, and is therefore a significant concern for public health. This dose was expected to result in 80%-100% mortality after intramuscular inoculation.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims should be construed to include all such embodiments and equivalent variations.

What is claimed is:

1. A pharmaceutical composition comprising a pharmaceutically acceptable carrier and at least two recombinant rabies virus-neutralizing human antibodies, wherein at least one of the at least two antibodies is selected from the group consisting of:
a) an antibody comprising an antibody light chain having the amino acid sequence SEQ ID NO:2 or a sequence that is substantially homologous to SEQ ID NO:2, and an antibody heavy chain having the amino acid sequence SEQ ID NO:1 or a sequence that is substantially homologous to SEQ ID NO:1;
b) an antibody comprising an antibody light chain having the amino acid sequence SEQ ID NO:6 or a sequence that is substantially homologous to SEQ ID NO:6, and an antibody heavy chain having the amino acid sequence SEQ ID NO:4 or a sequence that is substantially homologous to SEQ ID NO:4; and
c) an antibody comprising an antibody light chain having the amino acid sequence SEQ ID NO:7 or a sequence that is substantially homologous to SEQ ID NO:7, and an antibody heavy chain having the amino acid sequence SEQ ID NO:9 or a sequence that is substantially homologous to SEQ ID NO:9.

2. The pharmaceutical composition of claim 1, comprising:
a) an antibody comprising an antibody light chain having the amino acid sequence SEQ ID NO:2 or a sequence that is substantially homologous to SEQ ID NO:2, and an antibody heavy chain having the amino acid sequence SEQ ID NO:1 or a sequence that is substantially homologous to SEQ ID NO:1;
b) an antibody comprising an antibody light chain having the amino acid sequence SEQ ID NO:6 or a sequence that is substantially homologous to SEQ ID NO:6, and an antibody heavy chain having the amino acid sequence SEQ ID NO:4 or a sequence that is substantially homologous to SEQ ID NO:4; and
c) an antibody comprising an antibody light chain having the amino acid sequence SEQ ID NO:7 or a sequence that is substantially homologous to SEQ ID NO:7, and an antibody heavy chain having the amino acid sequence SEQ ID NO:9 or a sequence that is substantially homologous to SEQ ID NO:9.

3. The pharmaceutical composition of claim 2, comprising:
a) an antibody comprising an antibody light chain having the amino acid sequence SEQ ID NO:2 and an antibody heavy chain having the amino acid sequence SEQ ID NO:1;
b) an antibody comprising an antibody light chain having the amino acid sequence SEQ ID NO:6 and an antibody heavy chain having the amino acid sequence SEQ ID NO:4; and
c) an antibody comprising an antibody light chain having the amino acid sequence SEQ ID NO:7 and an antibody heavy chain having the amino acid sequence SEQ ID NO:9.

4. A method of treating or preventing a rabies virus infection in a subject in need of such treatment, comprising administering to the subject an effective amount of at least two recombinant rabies virus-neutralizing human antibodies, wherein at least one of the at least two antibodies is selected from the group consisting of:
a) an antibody comprising an antibody light chain having the amino acid sequence SEQ ID NO:2 or a sequence that is substantially homologous to SEQ ID NO:2, and an antibody heavy chain having the amino acid sequence SEQ ID NO:1 or a sequence that is substantially homologous to SEQ ID NO:1;
b) an antibody comprising an antibody light chain having the amino acid sequence SEQ ID NO:6 or a sequence that is substantially homologous to SEQ ID NO:6, and an antibody heavy chain having the amino acid sequence SEQ ID NO:4 or a sequence that is substantially homologous to SEQ ID NO:4; and
c) an antibody comprising an antibody light chain having the amino acid sequence SEQ ID NO:7 or a sequence that is substantially homologous to SEQ ID NO:7, and an antibody heavy chain having the amino acid sequence SEQ ID NO:9 or a sequence that is substantially homologous to SEQ ID NO:9.

5. The method of claim 4, wherein the at least two recombinant rabies virus-neutralizing human antibodies exhibit neutralizing activity against different rabies viruses.

6. The method of claim 5, wherein the at least two different recombinant rabies virus-neutralizing human antibodies are separately administered.

7. The method of claim 5, wherein at least three different recombinant rabies virus-neutralizing human antibodies are administered.

8. The method of claim 4, wherein the recombinant rabies virus-neutralizing human antibodies comprise:
a) an antibody comprising an antibody light chain having the amino acid sequence SEQ ID NO:2 or a sequence that is substantially homologous to SEQ ID NO:2, and an antibody heavy chain having the amino acid sequence SEQ ID NO:1 or a sequence that is substantially homologous to SEQ ID NO:1;
b) an antibody comprising an antibody light chain having the amino acid sequence SEQ ID NO:6 or a sequence that is substantially homologous to SEQ ID NO:6, and an antibody heavy chain having the amino acid sequence SEQ ID NO:4 or a sequence that is substantially homologous to SEQ ID NO:4; and
c) an antibody comprising an antibody light chain having the amino acid sequence SEQ ID NO:7 or a sequence that is substantially homologous to SEQ ID NO:7, and an antibody heavy chain having the amino acid sequence SEQ ID NO:9 or a sequence that is substantially homologous to SEQ ID NO:9.

9. The method of claim 8, wherein the recombinant rabies virus-neutralizing human antibodies comprise:
a) an antibody comprising an antibody light chain having the amino acid sequence SEQ ID NO:2 and an antibody heavy chain having the amino acid sequence SEQ ID NO:1;
b) an antibody comprising an antibody light chain having the amino acid sequence SEQ ID NO:6 and an antibody heavy chain having the amino acid sequence SEQ ID NO:4; and
c) an antibody comprising an antibody light chain having the amino acid sequence SEQ ID NO:7 and an antibody heavy chain having the amino acid sequence SEQ ID NO:9.

10. The method of claim 5, wherein the recombinant rabies virus-neutralizing human antibodies are administered in a mixture of approximately equimolar concentrations.

11. The method of claim 5, wherein the recombinant rabies virus-neutralizing human antibodies are administered in approximately equal amounts by weight.

12. The method of claim 11, wherein the amount of antibody administered is between about 0.001 mg/kg body weight and about 100 mg/kg body weight.

13. The method of claim 12, wherein the amount of antibody administered is between about 0.01 mg/kg body weight and about 60 mg/kg body weight.

14. The method of claim 5, wherein the at least three different recombinant rabies virus-neutralizing human antibodies comprise between about 1 IU/kg body weight and about 50 IU/kg body weight rabies virus-neutralizing activity.

15. The method of claim 5, wherein the rabies virus is a fixed rabies virus or a street rabies virus.

16. The method of claim 15, wherein the street rabies virus is selected from the group consisting of silver-haired bat rabies virus, coyote street rabies virus/Mexican dog rabies virus, and dog rabies virus.

17. The method of claim 16, wherein the silver-haired bat rabies virus is silver-haired bat rabies virus-18.

18. The method of claim 16, wherein the dog rabies virus is dog rabies virus-4.

19. The method of claim 5, wherein the subject is a human.

20. The method of claim 5, wherein the at least two recombinant rabies virus-neutralizing human antibodies are administered parenterally.

21. The method of claim 20, wherein the parenteral administration is selected from the group consisting of intravascular administration, peri- and intra-tissue injection, intraperitoneal injection, subcutaneous injection, subcutaneous deposition, and subcutaneous infusion.

22. A recombinant rhabdovirus expression vector, comprising: (i) a nucleic acid sequence encoding a vesicularstomatitisvirus glycoprotein sequence; and (ii) a nucleic acid sequence encoding an antibody light chain, or an antibody heavy chain, or both an antibody light chain and an antibody heavy chain, of a recombinant rabies virus-neutralizing human antibody.

23. The recombinant rhabdovirus expression vector of claim 22, wherein the vector further comprises a nucleic acid sequence encoding a promoter sequence operably linked to the (i) the nucleic acid sequence encoding a vesicularstomatitisvirus glycoprotein sequence; and (ii) the nucleic acid sequence encoding an antibody light chain, or an antibody heavy chain, or both an antibody light chain and an antibody heavy chain, of a recombinant rabies virus-neutralizing human antibody.

24. The recombinant rhabdovirus expression vector of claim 23, wherein the vector encodes an antibody light chain selected from the group consisting of:
a) an antibody light chain having the amino acid sequence SEQ ID NO:2 or a sequence that is substantially homologous to SEQ ID NO:2;
b) an antibody light chain having the amino acid sequence SEQ ID NO:6 or a sequence that is substantially homologous to SEQ ID NO:6; and
c) an antibody light chain having the amino acid sequence SEQ ID NO:7 or a sequence that is substantially homologous to SEQ ID NO:7.

25. The recombinant rhabdovirus expression vector of claim 23, wherein the vector encodes an antibody heavy chain selected from the group consisting of:
a) an antibody heavy chain having the amino acid sequence SEQ ID NO:1 or a sequence that is substantially homologous to SEQ ID NO:1;
b) an antibody heavy chain having the amino acid sequence SEQ ID NO:4 or a sequence that is substantially homologous to SEQ ID NO:4; and
c) an antibody heavy chain having the amino acid sequence SEQ ID NO:9 or a sequence that is substantially homologous to SEQ ID NO:9.

26. A host mammalian cell comprising a recombinant rhabdovirus expression vector selected from the group consisting of the recombinant rhabdovirus expression vectors according to claims 22, 23, 24, and 25.

27. The host mammalian cell of claim 26, wherein the mammalian cell is selected from the group consisting of BSR cells, baby hamster cells, VERO cells, and chinese hamster ovary cells.

28. A method of producing a recombinant rabies virus-neutralizing human antibody in a mammalian cell, comprising culturing a cell of claim 26, under conditions which allow expression of the recombinant rabies virus-neutralizing human antibody.

29. The method of claim 28, wherein the recombinant rabies virus-neutralizing human antibody is produced in a mammalian cell selected from the group consisting of BSR cells, baby hamster cells, VERO cells, and chinese hamster ovary cells.

30. Use of a combination of at least two recombinant rabies virus-neutralizing human antibodies, wherein at least one of the antibodies is selected from the group consisting of:
a) an antibody comprising an antibody light chain having the amino acid sequence SEQ ID NO:2 or a sequence that is substantially homologous to SEQ ID NO:2, and an antibody heavy chain having the amino acid sequence SEQ ID NO:1 or a sequence that is substantially homologous to SEQ ID NO:1;
b) an antibody comprising an antibody light chain having the amino acid sequence SEQ ID NO:6 or a sequence that is substantially homologous to SEQ ID NO:6, and an antibody heavy chain having the amino acid sequence SEQ ID NO:4 or a sequence that is substantially homologous to SEQ ID NO:4; and
c) an antibody comprising an antibody light chain having the amino acid sequence SEQ ID NO:7 or a sequence that is substantially homologous to SEQ ID NO:7, and an antibody heavy chain having the amino acid sequence SEQ ID NO:9 or a sequence that is substantially homologous to SEQ ID NO:9;
for preparation of a medicament for treating or preventing a rabies virus infection in a subject in need of such treatment.

31. Use of a combination according to claim 30, wherein the at least two recombinant rabies virus-neutralizing human antibodies exhibit neutralizing activity against different rabies viruses.

32. Use of a combination of antibodies according to claim 30, wherein the combination comprises at least three different recombinant rabies virus-neutralizing antibodies.

33. Use of a combination of antibodies according to claim 32, wherein the antibodies comprise:
a) an antibody comprising an antibody light chain having the amino acid sequence SEQ ID NO:2 or a sequence that is substantially homologous to SEQ ID NO:2, and an antibody heavy chain having the amino acid sequence SEQ ID NO:1 or a sequence that is substantially homologous to SEQ ID NO:1;
b) an antibody comprising an antibody light chain having the amino acid sequence SEQ ID NO:6 or a sequence that is substantially homologous to SEQ ID NO:6, and an antibody heavy chain having the amino acid sequence SEQ ID NO:4 or a sequence that is substantially homologous to SEQ ID NO:4; and
c) an antibody comprising an antibody light chain having the amino acid sequence SEQ ID NO:7 or a sequence that is substantially homologous to SEQ ID NO:7, and an antibody heavy chain having the amino acid sequence SEQ ID NO:9 or a sequence that is substantially homologous to SEQ ID NO:9.

34. Use of a combination of antibodies according to claim 33, wherein the antibodies comprise:
a) an antibody comprising an antibody light chain having the amino acid sequence SEQ ID NO:2 and an antibody heavy chain having the amino acid sequence SEQ ID NO:1;
b) an antibody comprising an antibody light chain having the amino acid sequence SEQ ID NO:6 and an antibody heavy chain having the amino acid sequence SEQ ID NO:4; and
c) an antibody comprising an antibody light chain having the amino acid sequence SEQ ID NO:7 and an antibody heavy chain having the amino acid sequence SEQ ID NO:9.


SEQUENCE LISTING (24 sequences)

SEQ ID NO: 1 - protein, 474 aa, Human [sequence omitted]

SEQ ID NO: 2 - protein, 234 aa, Human [sequence omitted]

SEQ ID NO: 3 - DNA, 1557 bp, Human
atggacacactttgctccacgctcctgctgctgaccatcccttcatgggtcttgtcccaaattaccttgaaggagactggtcctacgctggtgaaacccacacagaccctcacgctgacctgcaccttctcggggttctcactcagcactagtggagtgggtgtgggctggatccgtcagcccccaggaaaggccctggagtgggttacactcatttattgggatgatgataagcgttacagtccatctctggagaacagggtcaccatcaggaaggacacctccaaaaaccaggtggctcttacaatgacgaacatggaccctttggacacaggcacatactactgtgcgcacagacaacatatcagcagcttcccgtggttcgattcctggggccagggaaccctggtcaccgtctcctcagcttccaccaagggcccatcggtcttccccctggcgccctgctccaggagcacctctgggggcacagcggccctgggctgcctggtcaaggactacttccccgagccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacacctgcaacgtgaatcacaagcccagcaacaccaaggtggacaagagagttgagctcaaaaccccacttggtgacacaactcacacatgcccacggtgcccagagcccaaatcttgtgacacacctcccccgtgcccacggtgcccagagcccaaatcttgtgacacacctcccccgtgcccacggtgcccagagcccaaatcttgtgacacacctcccccatgcccacggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagttcaacagcacgttccgtgtggtcagcgtcctcaccgtcctgcaccaggactggctgaacggtaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatga

SEQ ID NO: 4 - protein, 518 aa, Human [sequence omitted]

SEQ ID NO: 5 - DNA, 699 bp, Human
atggcctggaccgttctcctcctcggcctcctctctcactgcacagggtctgtgacgtcctatgtgctgactcagccaccctcggtgtcagtggccccaggaaagacggccaggattaactgtgggggaaacaacattgaatatagaagtgtgcactggtaccagcagaagtcaggccaggcccctgtagcggtcatctatgataatagtgaccggccctcagggatccctgagcgattctctggttccaaatctgggaacacggccaccctgaccatcagcagggtcgaagccggggatgaggccgactattactgtcaggtgtgggatattagtagtgatgtggtcttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatag

SEQ ID NO: 6 - protein, 232 aa, Human [sequence omitted]

SEQ ID NO: 7 - protein, 242 aa, Human [sequence omitted]

SEQ ID NO: 8 - DNA, 1431 bp, Human
atggactggacctggaggttcctctttgtggtggcagcagctacaggtgtccagtcccaggtgcagctggtgcagtctggggctgaggtgaagaagcctgggtcctcggtgaaggtctcctgcaaggcttctggaggcaccttcaacaggtatactgtcaactgggtgcgacaggcccctggacaagggcttgagtggatgggaggcatcatccctatctttggtacagcaaactacgcacagaggttccagggcagactcaccattaccgcggacgaatccacgagcacagcctacatggagctgagcagcctgagatctgatgacacggccgtgtatttctgtgcgagagagaatctcgataattcggggacttattattatttctcaggctggttcgacccctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 9 - protein, 476 aa, Human [sequence omitted]

SEQ ID NO: 10 - DNA, 705 bp, Human
atggaagccccagctcagcttctcttcctcctgctactctggctcccagataccaccggagaaattgtgttgacacagtctccagccaccctgtctttgtctccaggggaaagagccaccctcgcctgcagggccagtcagactgctagcaggtacttagcctggtaccaacagaaacctggccaggctcccagactcctcatctatgatacatccaacagggccactggcatcccagccaggttcagtggcagtgggtctgggacagacttcactctctccatcagcagcctggagcctgaagattttgcagtttattactgtcagcagcgtttcaactggccgtggacgttcggccaagggaccaaggtggaattcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 11 - DNA, 1425 bp, Human
atggagtttgggctgagctggctttttcttgtggctattttaaaaggtgtccagtgtgaggtgcagctgttggagtctgggggaggcttggtacagcctggggggtccctgagactctcctgtgcagcctctggattcacctttagcaactatgccatgagctgggtccgccaggctccagggaaggggctggagtgggtctcagctattagtgctagtggtcatagcacatatttggcagactccgtgaagggccggttcaccatctccagagacaattccaagaacacgctgtatctgcaaatgaacagcctgagagccgaggacacggccgtatattactgtgcgaaagatcgagaggttactatgatagttgtacttaatggaggctttgactactggggccagggaacccgggtcaccgtctcctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 12 - DNA, 729 bp, Human
atgagtgtccccaccatggcctgggctctgctcctcctcagcctcctcactcagggcacaggatcctgggctcagtctgccctgactcagcctcgctcagtgtccgggtctcctggacagtcagtcaccatctcctgcactggaaccagcagtgatattggtggttataactttgtctcctggtaccaacaacacccaggcaaagcccccaaactcatgatttatgatgccactaagcggccctcaggggtccctgatcgcttctctggctccaagtctggcaacacggcctccctgaccatctctgggctccaggctgaggatgaggctgattattactgctgctcatatgcaggcgactacaccccgggcgtggttttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatag

SEQ ID NO: 13 - DNA, 33 bp, Artificial Sequence
  Primer
aaacgtacgatggagtttgggctgagctggctt

SEQ ID NO: 14 - DNA, 34 bp, Artificial Sequence
  Primer
aacgtacgatggacacactttgctccacgctcct

SEQ ID NO: 15 - DNA, 35 bp, Artificial Sequence
  Primer
aaacgtacgaccatggactggacctggaggttcct

SEQ ID NO: 16 - DNA, 49 bp, Artificial Sequence
  Primer
tgctaggggtgttagtttttttcatgactcatttacccggggacaggga

SEQ ID NO: 17 - DNA, 56 bp, Artificial Sequence
  Primer, where n is a 5′ end of light chain cDNAs
ggtaaatgagtcatgaaaaaaactaacacccctagcnnnnnnnnnnnnnnnnnnnn

SEQ ID NO: 18 - DNA, 34 bp, Artificial Sequence
  Primer
aaagctagcctaacactctcccctgttgaagctc

SEQ ID NO: 19 - DNA, 36 bp, Artificial Sequence
  Primer
aaagctagcctatgaacattctgtaggggccactgt

SEQ ID NO: 20 - DNA, 33 bp, Artificial Sequence
  Primer
aaatctagactatgaacattctgtaggggccac

SEQ ID NO: 21 - DNA, 29 bp, Artificial Sequence
  Primer
cctctagattacagtctggtctcaccccc

SEQ ID NO: 22 - DNA, 33 bp, Artificial Sequence
  Primer
cccgggttaacagaagagtcaatcgatcagaac

SEQ ID NO: 23 - DNA, 27 bp, Artificial Sequence
  Primer
ttaagttaaccaagaatagtccaatga

SEQ ID NO: 24 - DNA, 34 bp, Artificial Sequence
  Primer
tctcgagcccgggactatgaagtgccttttgtac